(19) United States
(12) Patent Application Publication
(10) Pub. No.: US 2025/0197417 A1
(43) Pub. Date: Jun. 19, 2025

(54) CRYSTALLINE INTERMEDIATES

(71) Applicant: QBIOTICS PTY LTD, Taringa, Queensland (AU)

(72) Inventors: Luca DOMENIGHINI, Milan (IT); Lorena FUMAGALLI, Milan (IT); Andrea GAMBINI, Milan (IT); Nicola SARDONE, Milan (IT)

(21) Appl. No.: 18/722,219

(22) PCT Filed: Dec. 21, 2022
(86) PCT No.: PCT/AU2022/051546

§ 371 Date: Jun. 20, 2024

(30) Foreign Application Priority Data
Dec. 21, 2021 (AU) 2021904153

(51) Int. Cl. C07D 493/06

(52) U.S. Cl. CPC C07D 493/06

(57) ABSTRACT

Provided herein is a crystalline intermediate useful in the manufacture of 6,7-epoxytigliane compounds. Methods of making the 6,7-epoxytigliane compounds using the crystalline intermediates are also provided, as well as high purity 6,7-epoxytigliane compounds that are able to be produced using the crystalline intermediate.

The present application claims priority from Australian provisional patent application no. 2021904153, filed on 21 Dec. 2021, the entire contents of which are incorporated herein by this reference.

### FIELD OF THE INVENTION

The present invention relates to a crystalline intermediate useful in the manufacture of 6,7-epoxytigliane compounds. Methods of making the 6,7-epoxytigliane compounds using the crystalline intermediates are also disclosed as well as high purity 6,7-epoxytigliane compounds that are able to be produced using the crystalline intermediate.

### BACKGROUND OF THE INVENTION

6,7-Epoxytiglianes are a class of diterpene esters that have been shown to have human and veterinary therapeutic application in diseases such as solid tumour cancers and bacterial infections as well as in promoting wound healing (WO2007070985, WO2014169356). A number of 6,7-epoxytigliane compounds are naturally occurring in plants belonging to Fontainea and Hylandia species, for example tigilanol tiglate (compound 1, 12-tigloyl-13-(2-methylbutanoyl)-6,7-epoxy-4,5,9,12,13,20-hexahydroxy-1-tigliaen-3-one, WO2007070985). Other epoxytigliane compounds are derived from naturally occurring 6,7-epoxytigliane compounds by a semi-synthetic process, for example 12,13-dihexanoyl-6,7-epoxy-4,5,9,12,13,20-hexahydroxy-1-tigliaen-3-one (compound 2, WO2014169356).

While at least some epoxytigliane compounds are available in therapeutic quantities from plant material, the extracts obtained contain multiple closely related epoxytigliane compounds that can be difficult to separate. Since variation in the 6,7-epoxytigliane compounds extracted occurs in the ester groups at the C12 and C13 positions of the 6,7-epoxytigliane structure, it has previously been proposed that the extract be treated to de-acylate the C12 and C13 esters to provide a single 6,7-epoxytigliane compound bearing hydroxy groups at the C12 and C13 positions. This intermediate compound may then be synthetically manipulated to produce a single desired natural or non-natural 6,7-epoxytigliane compound (WO2014169356).

However, 6,7-epoxytigliane compounds produced in this manner still require purification to ensure the removal of undesirable solvents and impurities that may be considered toxic. Some synthetic processes have been found to result in a small portion of the 6,7 epoxytigliane compound undergoing ring opening of the 6,7-epoxide ring to form hydrochloride adducts such as:

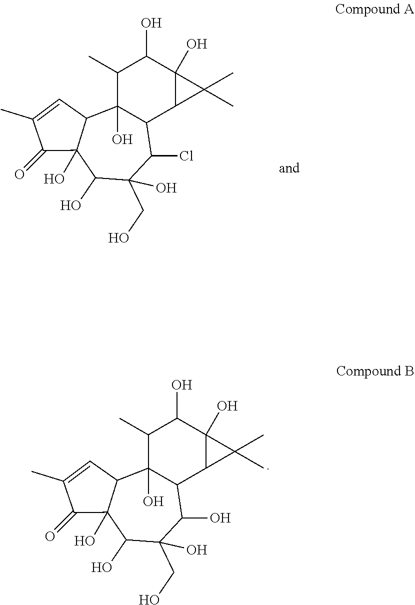

These compounds are considered toxic impurities and are difficult to remove from the desired 6,7-epoxytigliane compounds and result in loss of desired compound and low yields during chromatographic purification steps.

Although purification of the product 6,7-epoxytigliane compounds by crystallization has been attempted, these compounds appear particularly resistant to crystallization.

There is a need for methods of manufacturing 6,7-epoxytigliane compounds that result in high yields and high purity therapeutic product.

It is to be understood that, if any prior art publication is referred to herein, such reference does not constitute an admission that the publication forms a part of the common general knowledge in the art, in Australia or any other country.

### SUMMARY OF THE INVENTION

The present invention is based at least in part on the discovery that a key intermediate in the synthetic preparation of therapeutically useful 6,7-epoxytigliane compounds crystallizes to form a high purity product and allows the preparation of the desired 6,7-epoxytigliane compounds in high yield and high purity.

In a first aspect of the invention, there is provided a crystalline form of a compound of formula (I):

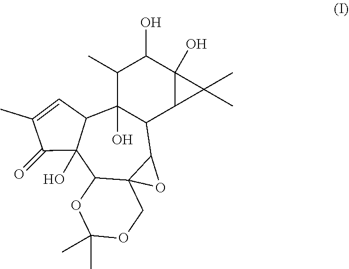

or a stereoisomer, pharmaceutically acceptable salt or solvate thereof.

In another aspect of the present invention there is provided method of making a crystalline form of a compound of formula (I) comprising the steps of:
i) providing a composition comprising one or more compounds of formula (II):

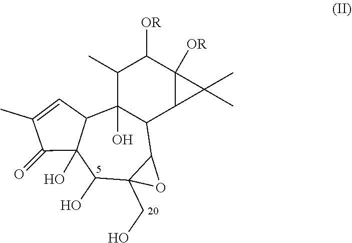

wherein each R is independently selected from H and —C(O)R₁, wherein when only one compound of formula (II) is present in the composition, at least one R group is not hydrogen; and
R₁ is selected from C₁-C₂₀alkyl, C₂-C₂₀alkenyl, C₂-C₂₀alkynyl, cycloalkyl, aryl, C₁₋₁₀alkylcycloalkyl; C₂₋₁₀alkenylcycloalkyl, C₂₋₁₀alkynylcycloalkyl, C₁₋₁₀alkylaryl, C₂₋₁₀alkenylaryl, C₂₋₁₀alkynylaryl, C₁₋₁₀alkylC(O)R₂, C₂₋₁₀alkenylC(O)R₂, C₂₋₁₀alkynylC(O)R₂, C₁₋₁₀alkylCH(OR₂)(OR₂), C(O)C₂₋₁₀alkenylCH(OR₂)(OR₂), C₂₋₁₀alkynylCH(OR₂)(OR₂), C₁₋₁₀alkylSR₂, C₂₋₁₀alkenylSR₂, C₂₋₁₀alkynylSR₂, C₁₋₁₀alkylC(O)OR₂, C₂₋₁₀alkenylC(O)OR₂, C₂₋₁₀alkynylC(O)OR₂, C₁₋₁₀alkylC(O)SR₂, C₂₋₁₀alkenylC(O)SR₂, C₂₋₁₀alkynylC(O)SR₂,

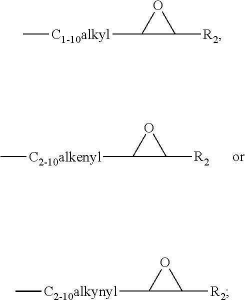

R₂ is hydrogen, —C₁₋₁₀alkyl, —C₂₋₁₀alkenyl, —C₂₋₁₀alkynyl, cycloalkyl or aryl; wherein each alkyl, alkenyl, alkynyl, cycloalkyl or aryl group is optionally substituted;
ii) forming a 5,20-acetonide of formula (III):

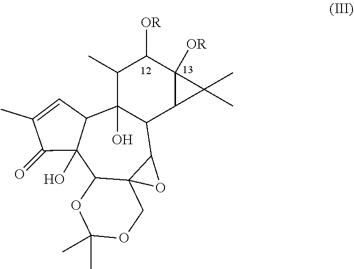

by treating the compound of formula (II) with 2,2-dimethoxypropane and a weakly acidic catalyst;
iii) de-esterifying the esters at C12 and C13 of formula (III) to provide a compound of formula (I):

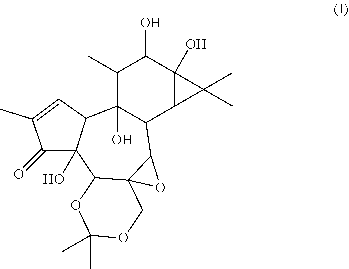

by treating the compound of formula (III) with a base; and
iv) crystallizing the compound of formula (I).

In yet a further aspect of the present invention, there is provided 12-tigloyl-13-(2-methylbutanoyl)-6,7-epoxy-4,5,9,12,13,20-hexahydroxy-1-tigliaen-3-one (compound 1) or 12,13-dihexanoyl-6,7-epoxy-4,5,9,12,13,20-hexahydroxy-1-tigliaen-3-one (compound 2) in substantially pure form.

In yet a further aspect of the present invention, there is provided a process for making a crystalline form of a compound of formula (I):

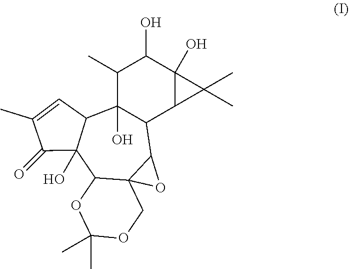

comprising crystallizing the compound of formula (I) from acetonitrile or methanol solvent.

### BRIEF DESCRIPTION OF THE FIGURES

FIG. 1 provides a representation of the X-ray powder diffraction pattern of anhydrous crystalline Form A of a compound of formula (Ia).

FIG. 2 provides a representation of the X-ray powder diffraction pattern of the anhydrous crystalline Form B of a compound of formula (Ia).

FIG. 3 provides a representation of the X-ray powder diffraction pattern of the methanol solvate of the compound of formula (Ia).

FIG. 4 provides a representation of the X-ray powder diffraction pattern of the dihydrate of the compound of formula (Ia).

### DETAILED DESCRIPTION OF THE INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 25%, 20%, 15% or 10% to a reference quantity, level, value, dimension, size, or amount. When used with respect to the position of a peak in an x-ray powder diffraction (XRPD) pattern, the term “about” include peaks within +0.2 degrees 2θ of the stated position, especially ±0.1 degrees 2θ of the stated position. For example, as used herein, an XRPD peak at “about 10.0 degrees 2θ” means that the stated peak occurs from 9.8 to 10.2 degrees 2θ.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The term “alkyl” refers to optionally substituted linear and branched saturated hydrocarbon groups having 1 to 20 carbon atoms. Where appropriate, the alkyl group may have a specified number of carbon atoms, for example, —C₁-C₆ alkyl which includes alkyl groups having 1, 2, 3, 4, 5 or 6 carbon atoms in linear or branched arrangements. Non-limiting examples of alkyl groups include methyl, ethyl, propyl, isopropyl, butyl, s- and t-butyl, pentyl, 2-methylbutyl, 3-methylbutyl, hexyl, 2-methylpentyl, 3-methylpentyl, 4-methylpentyl, 2-ethylbutyl, 3-ethylbutyl, heptyl, octyl, nonyl, decyl, undecyl, dodecyl, tridecyl, tetradecyl and pentadecyl.

The term “alkenyl” refers to optionally substituted, unsaturated linear or branched hydrocarbons, having 2 to 20 carbon atoms and having at least one double bond. Where appropriate, the alkenyl group may have a specified number of carbon atoms, for example, C₂-C₆ alkenyl which includes alkenyl groups having 2, 3, 4, 5 or 6 carbon atoms in linear or branched arrangements. Non-limiting examples of alkenyl groups include, ethenyl, propenyl, isopropenyl, butenyl, s- and t-butenyl, pentenyl, hexenyl, hept-1,3-diene, hex-1,3-diene, non-1,3,5-triene and the like.

The term “alkynyl” refers to optionally substituted unsaturated linear or branched hydrocarbons, having 2 to 20 carbon atoms, having at least one triple bond. Where appropriate, the alkynyl group may have a specified number of carbon atoms, for example, C₂-C₆ alkynyl which includes alkynyl groups having 2, 3, 4, 5 or 6 carbon atoms in linear or branched arrangements. Non-limiting examples include ethynyl, propynyl, butynyl, pentynyl and hexynyl.

The terms “cycloalkyl” and “carbocyclic” refer to optionally substituted saturated or unsaturated, but not aromatic, mono-cyclic, bicyclic or tricyclic hydrocarbon groups. Where appropriate, the cycloalkyl group may have a specified number of carbon atoms, for example, C₃-C₆ cycloalkyl is a carbocyclic group having 3, 4, 5 or 6 carbon atoms. Non-limiting examples may include cyclopropyl, cyclobutyl, cyclopentyl, cyclopentenyl, cyclohexyl, cyclohexenyl, cyclohexadienyl and the like.

“Aryl” means a C₆-C₁₄ membered monocyclic, bicyclic or tricyclic carbocyclic ring system having up to 7 atoms in each ring, wherein at least one ring is aromatic. Examples of aryl groups include, but are not limited to, phenyl, naphthyl, tetrahydronaphthyl, indanyl and biphenyl. The aryl may comprise 1-3 benzene rings. If two or more aromatic rings are present, then the rings may be fused together, so that adjacent rings share a common bond.

Each alkyl, alkenyl, alkynyl, cycloalkyl or aryl whether an individual entity or as part of a larger entity may be optionally substituted with one or more optional substituents selected from the group consisting of C₁₋₆alkyl, C₂₋₆alkenyl, C₂₋₆alkynyl, C₃₋₆cycloalkyl, oxo (═O), —OH, SH, OC₁₋₆alkyl, OC₂₋₆alkenyl, OC₂₋₆alkynyl, OC₃₋₆cycloalkyl, SC₁₋₆alkyl, SC₂₋₆alkenyl, SC₂₋₆alkynyl, SC₃₋₆cycloalkyl, CO₂H, CO₂C₁₋₆alkyl, NH₂, NH(C₁₋₆alkyl), N(C₁₋₆alkyl)₂, NH(phenyl), N(phenyl)₂, CN, NO₂, halogen, CF₃, —OCF₃, SCF₃, CHF₂, OCHF₂, SCHF₂, phenyl, C₁₋₆alkylphenyl, Ophenyl, C(O)phenyl and C(O)C₁₋₆alkyl. Examples of suitable substituents include, but are not limited to, methyl, ethyl, propyl, isopropyl, butyl, sec-butyl, tert-butyl, vinyl, methoxy, ethoxy, propoxy, isopropoxy, butoxy, methylthio, ethylthio, propylthio, isopropylthio, butylthio, hydroxy, hydroxymethyl, hydroxyethyl, hydroxypropyl, hydroxybutyl, fluoro, chloro, bromo, iodo, cyano, nitro, —CO₂H, —CO₂CH₃, —C(O)CH₃, trifluoromethyl, trifluoromethoxy, trifluoromethylthio, difluoromethyl, difluoromethoxy, difluoromethylthio, morpholino, amino, methylamino, dimethylamino, phenyl, phenoxy, phenylcarbonyl, benzyl and acetyl.

The epoxytigliane compounds may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternized with such agents as lower alkyl halide, such as methyl, ethyl, propyl, and butyl chlorides, bromides and iodides; dialkyl sulfates like dimethyl and diethyl sulfate; and others.

It will also be recognised that the epoxytigliane compounds may possess asymmetric centres and are therefore capable of existing in more than one stereoisomeric form. The invention thus also relates to compounds are substantially one stereoisomeric form at one or more asymmetric centres e.g., greater than about 90% ee, such as about 95% or 97% ee or greater than 99% ee, as well as mixtures, including racemic mixtures, thereof. Such stereoisomers may be obtained by isolation from natural sources, by asymmetric synthesis, for example using chiral intermediates, or by chiral resolution. The compounds of the invention may also exist as geometrical isomers. The invention also relates to compounds in substantially pure cis (Z) or trans (E) forms or mixtures thereof.

The term “6,7-epoxytigliane compound” refers to a compound having the following basic carbon cyclic structure:

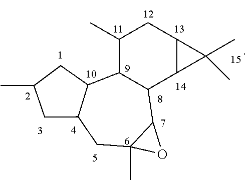

The compounds have a tricyclo[9.3.0.0]tetradecane system with a fused cyclopropane ring appended to the six membered ring. The epoxide is fused to the seven-membered ring in the 6,7-position.

One example of an 6,7-epoxytigliane compound is an 6,7-epoxytiglien-3-one compound. The term “epoxytiglien-3-one compound” refers to a compound having an epoxytigliane structure defined above where the five-membered ring has a 1,2-ene-3-one structure:

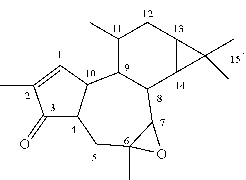

The term “substantially pure form” refers to a product having greater than 96% chemical purity, especially greater than 97%, greater than 98%, greater than 99% or 100% chemical purity and/or greater than 97% ee, especially greater than 98% or greater than 99% ee and more especially 100% ee. In some embodiments, substantially pure form refers to where impurity compounds A and/or B are present in less than 0.5%, less than 0.1%, less than 0.05% and most especially they are absent.

The present invention relates to a compound of formula (I) in crystalline form, especially a compound of formula (Ia) in crystalline form:

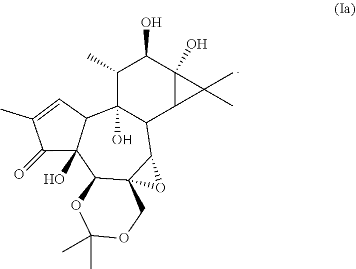

In some embodiments, the crystalline form is an anhydrous crystalline form. In other embodiments, the crystalline form is a methanol solvate. In another embodiment, the crystalline form is a dihydrate.

In embodiments, where the crystalline form is an anhydrous crystalline form of a compound of formula (Ia), the crystalline form may be one of two forms.

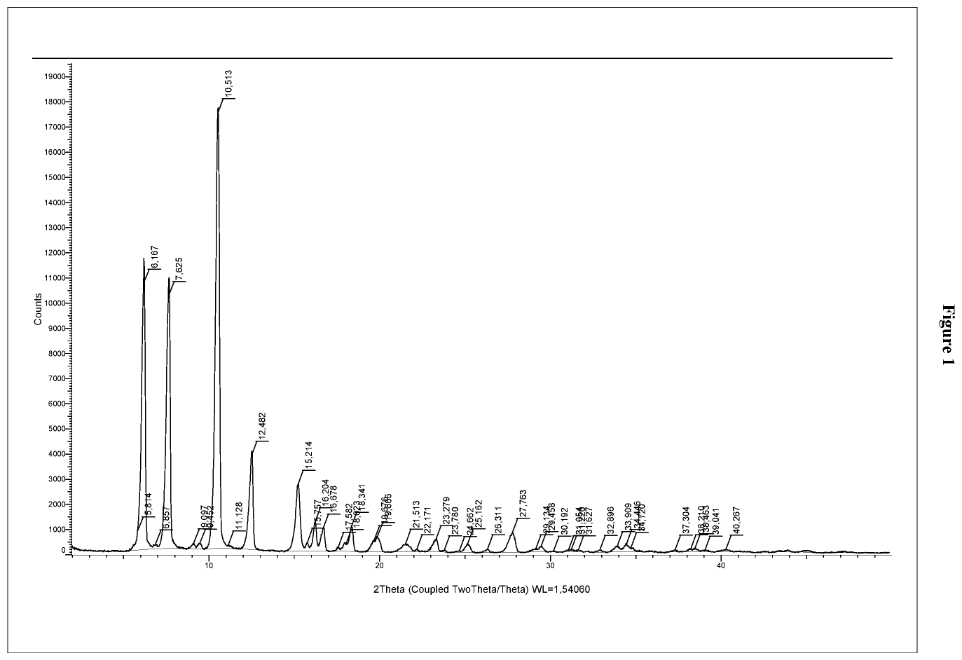

Form A of the anhydrous crystalline form exhibits an X-ray powder diffraction (XRPD) pattern comprising at least one peak at about 10.5 degrees 2θ. In another embodiment, anhydrous crystalline Form A exhibits an XRPD pattern comprising a peak at about 10.5 degrees 2θ and at least one peak selected from the group consisting of about 6.2 and about 7.6 degrees 2θ. In another embodiment, the anhydrous crystalline Form A exhibits an XRPD pattern comprising a peak at about 10.5 degrees 2θ, at least one peak selected from about 6.2 and about 7.6 degrees 2θ and at least one peak selected from about 12.5 and about 15.2 degrees 2θ. In yet a further embodiment, the anhydrous crystalline Form A exhibits an XRPD pattern comprising a peak at about 10.5 degrees 2θ, at least one peak selected from about 6.2 and about 7.6 degrees 2θ, at least one peak selected from about 12.5 and about 15.2 degrees 2θ and at least one peak selected from about 16.2, about 16.7, about 18.3, about 19.9, about 23.3 and about 27. 8 degrees 2θ. In one embodiment, the XRPD pattern is substantially the same as FIG. 1. In another embodiment, the anhydrous crystalline Form A has an XRPD pattern with peaks at 6.2, 7.6, 10.5, 12.5, 15.2, 16.2, 16.7, 18.3, 19.9, 21.5, 23.3, 25.2 and 27.8±0.2 degrees 2θ.

Furthermore, the anhydrous crystalline Form A is characterized by a Fourier Transform Infra-red (FTIR) spectrum in the 4000-550 cm⁻¹ spectral range in attenuated total reflectance (ATR) mode comprising absorption frequencies at about 3458.4, 3345.6, 3056.9, 2984.6, 2968.6, 2941.1, 2882.4, 1708.3, 1636.1, 1458.5, 1376.1, 1319.0, 1223.5, 1085.7, 1025.4, 1000.3, 973.7, 930.3, 902.1, 880.3, 832.1 and 769.0 cm⁻¹.

In some embodiments, the anhydrous crystalline Form A has a water content of less than 0.5%, especially less than 0.3%, for example about 0.28%.

In some embodiments, the anhydrous crystalline Form A has a level of residual organic solvent of less than 50 ppm, especially less than 40 ppm and more especially less than 25 ppm, for example, about 34 ppm.

A second anhydrous crystalline form (Form B) may be formed from anhydrous crystalline Form A under high humidity conditions, for example, a temperature of 25° C. and a relative humidity of 80% over 68 hours. Anhydrous crystalline Form B is stable upon storage and may be present upon storage of anhydrous crystalline Form A.

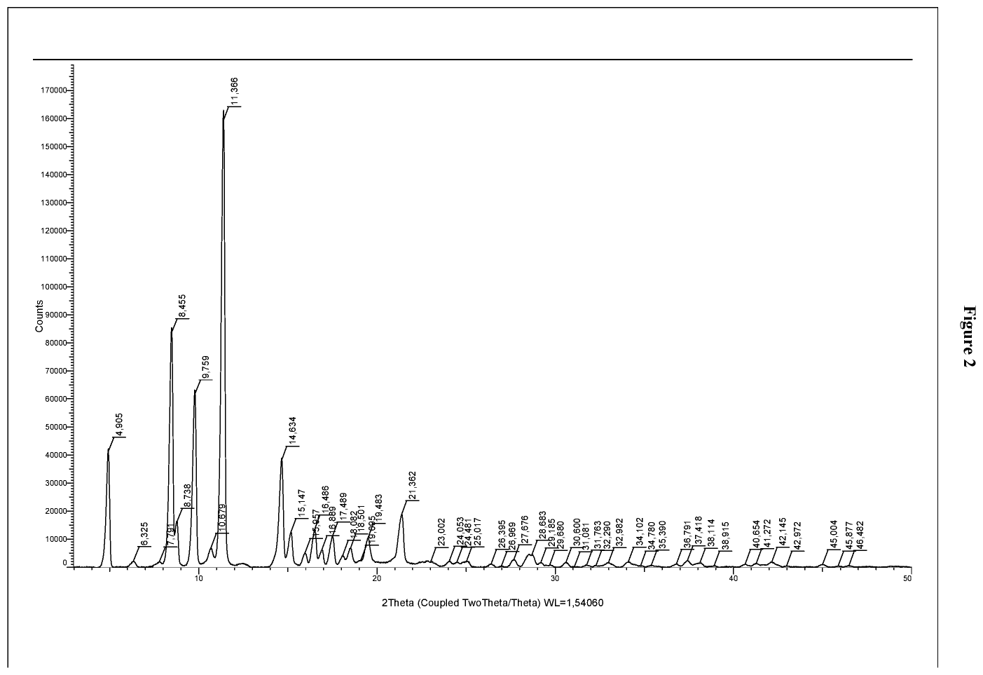

Form B of the anhydrous crystalline form exhibits an X-ray powder diffraction (XRPD) pattern comprising at least one peak at about 11.4 degrees 2θ. In another embodiment, anhydrous crystalline Form B exhibits an XRPD pattern comprising a peak at about 11.4 degrees 2θ and at least one peak selected from the group consisting of about 8.5 and about 9.8 degrees 2θ. In another embodiment, the anhydrous crystalline Form B exhibits an XRPD pattern comprising a peak at about 11.4 degrees 2θ, at least one peak selected from about 8.5 and about 9.8 degrees 2θ and at least one peak selected from about 4.9 and about 14.6 degrees 2θ. In yet a further embodiment, the anhydrous crystalline Form B exhibits an XRPD pattern comprising a peak at about 11.4 degrees 2θ, at least one peak selected from about 8.5 and about 9.8 degrees 2θ, at least one peak selected from about 4.9 and about 14.6 degrees 2θ and at least one peak selected from about 16.5, about 17.5, about 19.5, about 21.4, about 27.7 and about 28.7 degrees 2θ. In one embodiment, the XRPD pattern is substantially the same as FIG. 2. In another embodiment, the anhydrous crystalline Form B has an XRPD pattern with peaks at 4.9, 8.5, 9.8, 11.4, 14.6, 16.5, 17.5, 19.5, 21.4, 27.7, 28.7±0.2 degrees 2θ.

In some embodiments, the anhydrous crystalline Form B has a water content of less than 0.5%, especially less than 0.3%, for example about 0.28%.

In some embodiments, the anhydrous crystalline Form B has a level of residual organic solvent of less than 50 ppm, especially less than 40 ppm and more especially less than 25 ppm, for example, about 34 ppm.

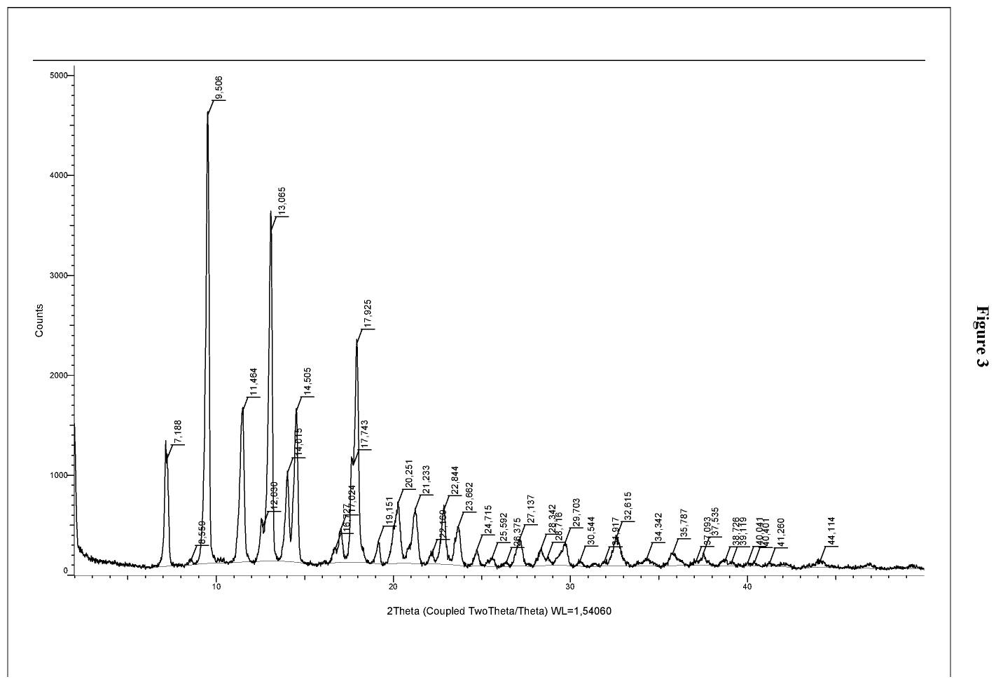

In embodiments where the crystalline form is a methanol solvate crystalline form of a compound of formula (Ia), the crystalline form exhibits an X-ray powder diffraction (XRPD) pattern comprising at least one peak at about 9.5 degrees 2θ. In another embodiment, the crystalline form exhibits an XRPD pattern comprising a peak at about 9.5 degrees 2θ and at least one peak selected from the group consisting of about 7.2 and about 13.1 degrees 2θ. In another embodiment, the crystalline form exhibits an XRPD pattern comprising a peak at about 9.5 degrees 2θ, at least one peak selected from about 7.2 and about 13.1 degrees 2θ and at least one peak selected from about 11.5, about 14.5 and about 17.9 degrees 2θ. In yet a further embodiment, the crystalline form exhibits an XRPD pattern comprising a peak at about 9.5 degrees 2θ, at least one peak selected from about 7.2 and about 13.1 degrees 2θ, at least one peak selected from about 11.5, about 14.5 and about 17.9 degrees 2θ and at least one peak selected from about 20.3, about 21.2, about 22.8 and about 32.6 degrees 2θ. In one embodiment, the XRPD pattern is substantially the same as FIG. 3. In another embodiment, the methanol solvate crystalline form has an XRPD pattern with peaks at 7.2, 9.5, 11.5, 12.6, 13.1, 14.0, 14.5, 17.0, 17.7, 17.9, 19.2, 20.3, 21.2, 22.8, 23.7, 24.7, 27.1, 28.3, 29.7 and 32.6±0.2 degrees 2θ.

Furthermore, the crystalline methanol solvate is characterised by a Fourier Transform Infra-red (FTIR) spectrum in the 4000-550 cm¹ spectral range in attenuated total reflectance (ATR) mode comprising absorption frequencies at about 3443.1, 3374.1, 3189.1, 3002.6, 2975.7, 2944.4, 2863.6, 1703.3, 1635.7, 1461.9, 1372.9, 1290.7, 1221.1, 1077.1, 1025.1, 992.0, 973.9, 929.7, 905.8, 876.7, 829.0 and 769.1.

In some embodiments, the crystalline methanol solvate has a differential scanning calorimetry (DSC) profile characterised by an endothermic peak having an onset at about 145.33° C. and in some embodiments one or two less intense endothermic peaks at about 221.12° C. and/or 234.22° C.

In some embodiments, the crystalline methanol solvate comprises 4 to 8%, especially 5 to 6% residual methanol as determined by thermogravimetric analysis.

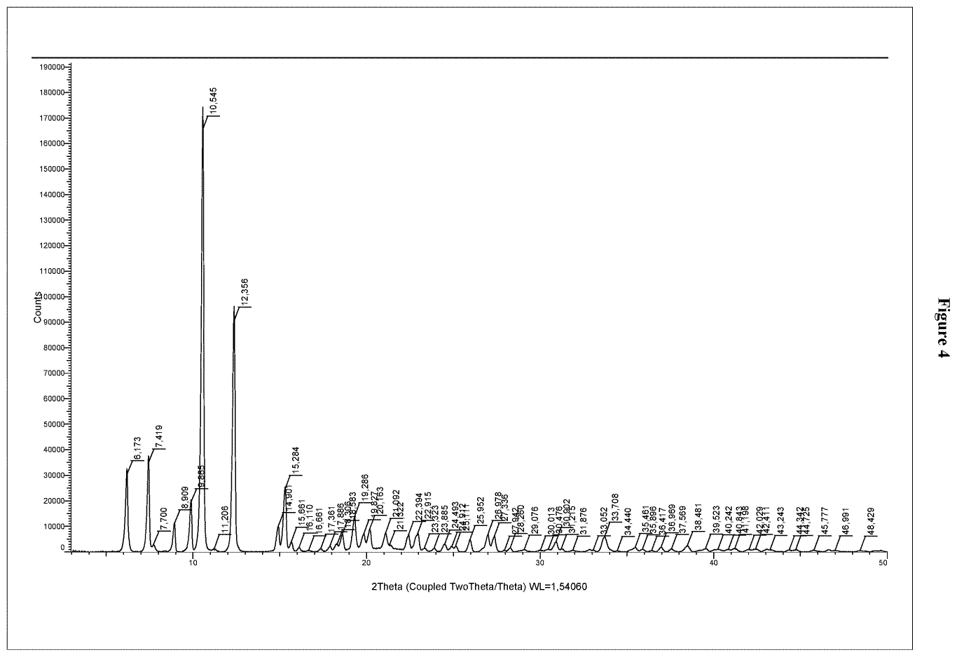

In embodiments where the crystalline form is a dihydrate crystalline form of a compound of formula (Ia), the crystalline form exhibits an X-ray powder diffraction (XRPD) pattern comprising at least one peak at about 10.5 degrees 2θ. In another embodiment, the crystalline form exhibits an XRPD pattern comprising a peak at about 10.5 degrees 2θ and at least one peak selected from the group consisting of about 7.4 and about 12.4 degrees 2θ. In another embodiment, the crystalline form exhibits an XRPD pattern comprising a peak at about 10.5 degrees 2θ, at least one peak selected from about 7.4 and about 12.4 degrees 2θ and at least one peak selected from about 6.2, about 9.9 and about 15.3 degrees 2θ. In yet a further embodiment, the crystalline form exhibits an XRPD pattern comprising a peak at about 10.5 degrees 2θ, at least one peak selected from about 7.4 and about 12.4 degrees 2θ, at least one peak selected from about 6.2, about 9.9 and about 15.3 degrees 2θ and at least one peak selected from about 8.9, about 18.6, about 20.2, about 22.4, about 22.9, about 26.0, about 27.0, about 27.3 and about 33.7 degrees 2θ. In one embodiment, the XRPD pattern is substantially the same as FIG. 4.

In another embodiment, the dihydrate crystalline form has an XRPD pattern with peaks at 6.2, 7.4, 8.9, 9.9, 10.5, 12.4, 15.3, 18.6, 20.2, 21.1, 22.4, 22.9, 26.0, 27.0, 27.3 and 33.7±0.2 degrees 2θ.

The dihydrate crystalline form may be formed upon storage at low temperature, such as about 5° C., and may be converted back to anhydrous Form A or anhydrous Form B by storage at room temperature and 80% humidity.

In some embodiments, the crystalline form is substantially pure, for example, having greater than 96%, 97%, 98% or 99% chemical purity, especially between 99% and 100% chemical purity, more especially between 99.5% and 100% chemical purity and optionally greater than 97% ee, especially greater than 98% ee or greater than 99% ee and more especially 100% ee.

In some embodiments, the crystalline form is a single crystalline form, for example, Form A or Form B of the anhydrous crystalline form. In other embodiments, the crystalline form is a mixture of crystalline forms, for example, a mixture of Form A and Form B or a mixture of From A and the dihydrate crystalline solvate. However, even if multiple crystalline forms are present, the compound of formula (Ia) may still be substantially pure having greater than 96%, 97%, 98% or 99% chemical purity, especially between 99% and 100% chemical purity, more especially between 99.5% and 100% chemical purity and optionally greater than 97% ee, especially greater than 98% ee or greater than 99% ee and more especially 100% ee. In particular embodiments, the crystalline form of the compound of formula (I) is an anhydrous crystalline form, especially the anhydrous crystalline form of the compound of formula (Ia):

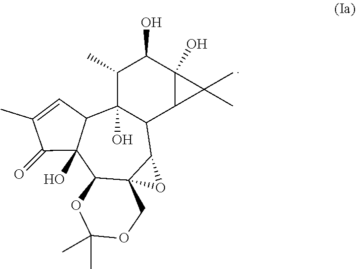

In some embodiments, the anhydrous crystalline form is Form A. In other embodiments, the anhydrous crystalline form is Form B.

The crystalline forms of the invention may be prepared by crystallization of a compound of formula (I) from a suitable solvent. Suitable solvents were investigated as shown in Example 7.

Crystallization from methanol provided the crystalline methanol solvate. Crystallization occurred after dissolution of the compound in methanol, suitably 2 to 10 volumes (2V to 10V) compared to the weight of compound, especially 3V to 8 V, more especially 3 V to 7V and more especially about 3V to 6V at 20 to 25° C. Crystallization occurred spontaneously after dissolution.

Crystallization from acetonitrile provided the crystalline anhydrous form, Form A, via a 1:1 acetonitrile (MeCN) solvate. Dissolution of the compound of formula (I) was obtained at reflux, with crystallization occurring upon cooling to about 68 to 70° C. Crystallization occurred in ratios of 3V to 15V of solvent compared to the weight of compound, especially 4V to 12V and more especially 5V to 10V. The initial MeCN solvate was collected by filtration and upon drying yielded the anhydrous crystalline Form A.

Advantageously the anhydrous crystalline form, Form A, was free of solvents and provided higher yields, 85-95% yield, compared to the crystalline methanol solvate, 70-80% yield.

Although the anhydrous crystalline Form A was stable at 60% relative humidity, at higher humidity, such as 80% relative humidity was able to transform to a dihydrate (storage at about 5° C.) or a second anhydrous crystalline form, Form B, (storage at about 25° C.).

### Manufacture of Compounds of Formula (I) in Anhydrous or Solvate Form

Compounds of formula (I) may be manufactured as set out in WO2014/169356 (Example 3) as shown in Scheme 1:

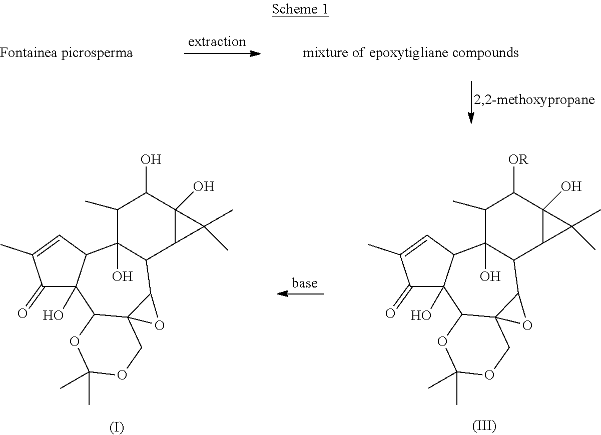

In one aspect of the present invention there is provided a method of making a crystalline form of a compound of formula (I) comprising the steps of:
i) providing a composition comprising one or more compounds of formula (II):

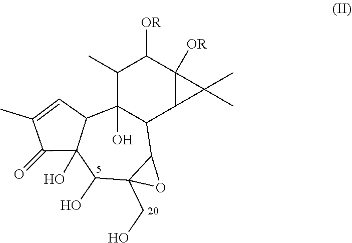

wherein each R is independently selected from H and —C(O)R₁, wherein when only one compound of formula (II) is present in the composition, at least one R group is not hydrogen; and
R₁ is selected from C₁-C₂₀alkyl, C₂-C₂₀alkenyl, C₂-C₂₀alkynyl, cycloalkyl, aryl, C₁₋₁₀alkylcycloalkyl; C₂₋₁₀alkenylcycloalkyl, C₂₋₁₀alkynylcycloalkyl, C₁₋₁₀alkylaryl, C₂₋₁₀alkenylaryl, C₂₋₁₀alkynylaryl, C₁₋₁₀alkylC(O)R₂, C₂₋₁₀alkenylC(O)R₂, C₂₋₁₀alkynylC(O)R₂, C₁₋₁₀alkylCH(OR₂)(OR₂), C(O)C₂₋₁₀alkenylCH(OR₂)(OR₂), C₂₋₁₀alkynylCH(OR₂)(OR₂), C₁₋₁₀alkylSR₂, C₂₋₁₀alkenylSR₂, C₂₋₁₀alkynylSR₂, C₁₋₁₀alkylC(O)OR₂, C₂₋₁₀alkenylC(O)OR₂, C₂₋₁₀alkynylC(O)OR₂, C₁₋₁₀alkylC(O)SR₂, C₂₋₁₀alkenylC(O)SR₂, C₂₋₁₀alkynylC(O)SR₂,

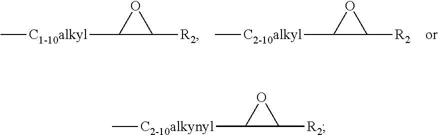

R₂ is hydrogen, —C₁₋₁₀alkyl, —C₂₋₁₀alkenyl, —C₂₋₁₀alkynyl, cycloalkyl or aryl; wherein each alkyl, alkenyl, alkynyl, cycloalkyl or aryl group is optionally substituted;
ii) forming a 5,20-acetonide of formula (III):

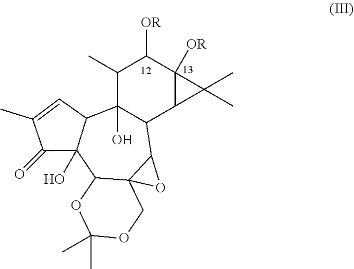

by treating the compound of formula (II) with 2,2-dimethoxypropane and a weakly acidic catalyst;
iii) de-esterifying the esters at C12 and C13 of formula (III) to provide a compound of formula (I):

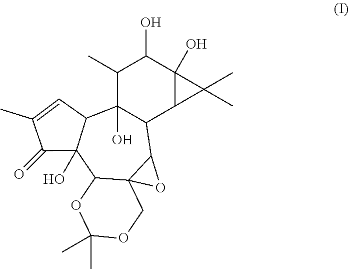

by treating the compound of formula (III) with a base; and crystallizing the compound of formula (I).

In some embodiments, the composition comprising one or more compounds of formula (II) comprises a mixture of compounds of formula (II), especially a mixture of compounds of formula (IIa):

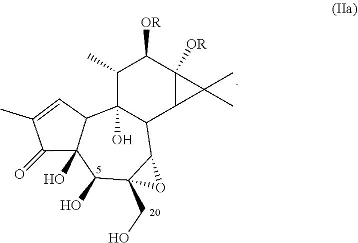

For example, the composition comprising one or more compounds of formula (II) or (IIa) comprises one or more compounds selected from the following:

| Compound | name |
|---|---|
| 1 | 12-tigloyl-13-(2-methylbutanoyl)-6,7-epoxy-4,5,9,12,13,20- |
| | hexahydroxy-1-tigliaen-3-one |
| 2 | 12,13-dihexanoyl-6,7-epoxy-4,5,9,12,13,20-hexahydroxy-1-tigliaen-3- |
| | one |
| 3 | 12,13-di-(2-methylbutanoyl)-6,7-epoxy-4,5,9,12,13,20-hexahydroxy-1- |
| | tigliaen-3-one |
| 4 | 12-[(2E,4E,6E)-dodeca-2,4,6-trienoyl]-13-(2-methylbutanoyl)-6,7-epoxy- |
| | 4,5,9,12,13,20- hexahydroxy-1-tigliaen-3-one |
| 5 | 12-[(2E,4Z)-deca-2,4-dienoyl]-13-(2-methylbutanoyl)-6,7-epoxy- |
| | 4,5,9,12,13,20-hexahydroxy-1-tigliaen-3-one |
| 6 | 12-hexanoyl-13-(2-methylbutanoyl)-6,7-epoxy-4,5,9,12,13,20- |
| | hexahydroxy-1-tigliaen-3-one |
| 7 | 12-acetyl-13-(2-methylbutanoyl)-6,7-epoxy-4,5,9,12,13,20-hexahydroxy- |
| | 1-tigliaen-3-one |
| 8 | 12-propanoyl-13-(2-methylbutanoyl)-6,7-epoxy-4,5,9,12,13,20- |
| | hexahydroxy-1-tigliaen-3-one |
| 9 | 12-butanoyl-13-(2-methylbutanoyl)-6,7-epoxy-4,5,9,12,13,20- |
| | hexahydroxy-1-tigliaen-3-one |
| 10 | 12-[(2E,4E)-(6,6-dimethoxyhexa-2,4-dienoyl]-13-(2-methylbutanoyl)- |
| | 6,7-epoxy-4,5,9,12,13,20-hexahydroxy-1-tigliaen-3-one |
| 11 | 12-[(2E,4E)-6-oxohexa-2,4-dienoyl]-13-(2-methylbutanoyl)-6,7-epoxy- |
| | 4,5,9,12,13,20-hexahydroxy-1-tigliaen-3-one |
| 12 | 12-[(2E,4E)-6,7-dihydroxydodeca-2,4-dienoyl]-13-(2-methylbutanoyl)- |
| | 6,7-epoxy-4,5,9,12,13,20-hexahydroxy-1-tigliaen-3-one |
| 13 | 12-[(2E)-4,5-dihydroxy-deca-2-enoyl]-13-(2-methylbutanoyl)-6,7-epoxy- |
| | 4,5,9,12,13,20-hexahydroxy-1-tigliaen-3-one |
| 14 | 12-tigloyl-13-(2-methylpropanoyl)-6,7-epoxy-4,5,9,12,13,20- |
| | hexahydroxy-1-tigliaen-3-one |
| 15 | 12-[(2E)-3-methylthioprop-2-enoyl]-13-(2-methylbutanoyl)-6,7-epoxy- |
| | 4,5,9,12,13,20-hexahydroxy-1-tigliaen-3-one |
| 16 | 12-(2-methylprop-2-enoyl-13-(2-methylbutanoyl)-6,7-epoxy- |
| | 4,5,9,12,13,20-hexahydroxy-1-tigliaen-3-one |
| 17 | 12-[(2E,4E)-hexa-2,4-dienoyl]-13-(2-methylbutanoyl)-6,7-epoxy- |
| | 4,5,9,12,13,20-hexahydroxy-1-tigliaen-3-one |
| 18 | 12-[(2E,4E)-8-oxododeca-2,4-dienoyl]-13-(2-methylbutanoyl)-6,7- |
| | epoxy-4,5,9,12,13,20-hexahydroxy-1-tigliaen-3-one |
| 19 | 12-[(2Z,4E)-deca-2,4-dienoyl]-13-(2-methylbutanoyl)-6,7-epoxy- |
| | 4,5,9,12,13,20-hexahydroxy-1-tigliaen-3-one |
| 20 | 13-(2-methylbutanoyl)-6,7-epoxy-4,5,9,12,13,20-hexahydroxy-1- |
| | tigliaen-3-one |
| 21 | 12-[(2E)-but-2-enoyl]-13-(2-methylbutanoyl)-6,7-epoxy-4,5,9,12,13,20- |
| | hexahydroxy-1-tigliaen-3-one |
| 22 | 12-[(2E,4E)-deca-2,4-dienoyl]-13-(2-methylbutanoyl)-6,7-epoxy- |
| | 4,5,9,12,13,20-hexahydroxy-1-tigliaen-3-one |
| 23 | 12-[(2Z,4E)-deca-2,4-dienoyl]-13-(2-methylpropanoyl)-6,7-epoxy- |
| | 4,5,9,12,13,20-hexahydroxy-1-tigliaen-3-one |
| 24 | 12-[(2E,4E)-6,7-(anti)-epoxy-dodeca-2,4-dienoyl]-13-(2- |
| | methylbutanoyl)-6,7-epoxy-4,5,9,12,13,20-hexahydroxy-1-tigliaen-3-one |

or a stereoisomer thereof, especially a stereoisomer having stereocentres as shown in formula (IIa).

In some embodiments, the weakly acidic catalyst is for example, a weak Brønsted acid catalyst such as pyridinium p-toluene sulfonic acid, or camphorsulfonic acid (CSA), a cation exchange resin, p-toluene sulfonic acid, H₂SO₄, zinc chloride and other Lewis acids, and BF₃ etherate. In particular embodiments, the weakly acidic catalyst is pyridinium p-toluene sulfonic acid.

The solvent used in the formation of the acetonide is any suitable solvent that is able to solubilise the reactants. Suitable solvents include acetone, dimethyl formamide, toluene, dichloromethane, tetrahydrofuran (THF), 2-methyltetrahydrofuran and acetonitrile.

De-esterification of the C12 and C13 esters occurs in the presence of a base. Suitable bases include carbonates such as Cs₂CO₃, Na₂CO₃, K₂CO₃ and Li₂CO₃. In particular embodiments, the base used ins Cs₂CO₃ in an aqueous or alcoholic solvent or in dimethylformamide (DMF), especially Cs₂CO₃ in methanol.

In some embodiments, the 5,20-acetonide compound of formula (I) is purified by column chromatography before crystallization. In other embodiments, the compound of formula (I) is crystallized without chromatographic purification.

Crystallization of the compound of formula (I) may be from a suitable solvent such as methanol or acetonitrile. From methanol solvents, the methanol solvate crystalline form was provided.

From acetonitrile, the crystalline acetonitrile solvate, crystalline anhydrous forms, Form A and Form B and the dihydrate crystalline form were provided. In particular embodiments, the crystalline forms are crystalline forms of the compound of formula (Ia).

One or more crystalline forms of the compound of formula (I) may be used to prepare 6.7-epoxytigliane compounds of therapeutic value, such as compound 1 and compound 2, in high yield and high purity.

Use of the one or more crystalline forms of the compound of formula (I) in the synthesis of compounds of 6,7-epoxytigliane compounds may improve the overall yield obtained by up to 50% and/or may reduce impurities, such as solvents and/or hydrochloride adducts A and B, to therapeutically acceptable levels.

In some embodiments, the method of the invention further comprises the step of esterifying one or both of the C12 and C13 hydroxy groups of the compound of formula (I) as shown in scheme 2 where the starting material is the crystalline compound of formula (I):

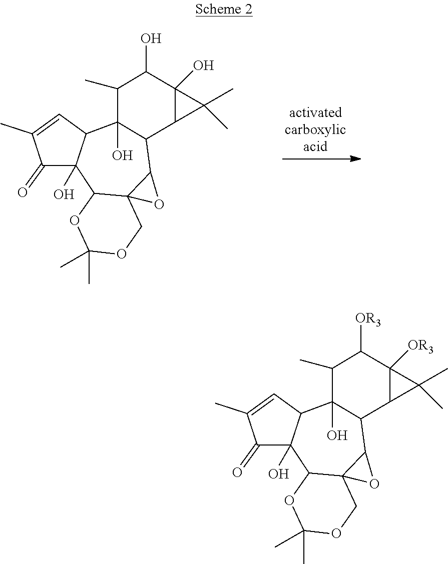

The reaction shown in scheme 2 may be undertaken in a single step where esterification of both the C12 and C13 hydroxy groups occurs with a single activated carboxylic acid. In this embodiment, each R₃ is the same.

In other embodiments, the C12 and C13 hydroxy groups are esterified in separate sequential reactions to produce a 6,7-epoxytigliane compound having different C12 an C13 esters where each R₃ is different. During the reaction, the first esterification occurs at the C13 hydroxy group and the reaction may be monitored to ascertain when reaction at C13 is complete.

The carboxylic acid may be activated for reaction with the hydroxy group by any means known in the art. In some embodiments, the activated carboxylic acid is an acid chloride or an anhydride. In some embodiments, the activated carboxylic acid may be a symmetric anhydride such as tiglic anhydride. In other embodiments, the activated carboxylic acid may be an unsymmetrical anhydride, for example, tiglic-pivalic anhydride. In yet other embodiments, the carboxylic acid is activated with a carbodiimide, for example, suitable carbodiimides include N,N′-dicyclohexylcarbodiimide (DCC), N,N′-diisopropylcarbodiimide (DIC) or 1-ethyl-3-(3-dimethylaminopropyl)carbodiimide (EDC) and the like.

Each R₃ is the same or different and is independently selected from the group consisting of H and —C(O)R₁ and R₁ is selected from C₁-C₂₀alkyl, C₂-C₂₀alkenyl, C₂-C₂₀alkynyl, cycloalkyl, aryl, C₁₋₁₀alkylcycloalkyl; C₂₋₁₀alkenylcycloalkyl, C₂₋₁₀alkynylcycloalkyl, C₁₋₁₀alkylaryl, C₂₋₁₀alkenylaryl, C₂₋₁₀alkynylaryl, C₁₋₁₀alkylC(O)R₂, C₂₋₁₀alkenylC(O)R₂, C₂₋₁₀alkynylC(O)R₂, C₁₋₁₀alkylCH(OR₂)(OR₂), C(O)C₂₋₁₀alkenylCH(OR₂)(OR₂), C₂₋₁₀alkynylCH(OR₂)(OR₂), C₁₋₁₀alkylSR₂, C₂₋₁₀alkenylSR₂, C₂₋₁₀alkynylSR₂, C₁₋₁₀alkylC(O)OR₂, C₂₋₁₀alkenylC(O)OR₂, C₂₋₁₀alkynylC(O)OR₂, C₁₋₁₀alkylC(O)SR₂, C₂₋₁₀alkenylC(O)SR₂, C₂₋₁₀alkynylC(O)SR₂,

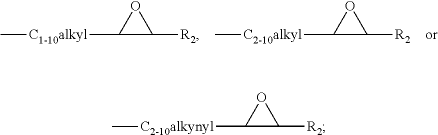

R₂ is hydrogen, —C₁₋₁₀alkyl, —C₂₋₁₀alkenyl, —C₂₋₁₀alkynyl, cycloalkyl or aryl;
wherein each alkyl, alkenyl, alkynyl, cycloalkyl or aryl group is optionally substituted. In particular embodiments, at least one of R₃ is other than hydrogen, especially where both R₃ are other than hydrogen.

Once the C12 and C13 esters are formed in the desired manner, the 5,20-acetonide protecting group may be removed to form the epoxytigliane compound of formula (IV) as shown in Scheme 3:

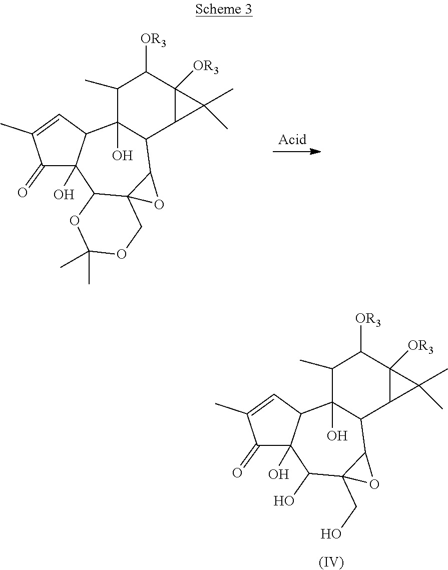

The acetonide protecting group may be removed under any suitable conditions as known in the art, for example, aqueous acidic conditions. Suitable acids that may be used to provide the acidic conditions include hydrochloric acid (HCl), trifluoroacetic acid, perchloric acid, sulfuric acid, Amberlite™ resin or other strong ion exchange resins and Lewis acids such as BF₃ etherate. Suitable solvents include aqueous and alcoholic solvents such as water, methanol, aqueous methanol, aqueous tetrahydrofuran, aqueous acetonitrile and the like. Examples of suitable conditions include HCl in aqueous methanol or aqueous tetrahydrofuran, trifluoroacetic acid in aqueous acetonitrile or aqueous perchloric acid (HClO₄).

In one embodiment, the method produces 12,13-dihexanoyl-6,7-epoxy-4,5,9,12,13,20-hexahydroxy-1-tigliaen-3-one (compound 2) in high yield and/or in substantially pure form.

In another embodiment, the method produces 12-tigloyl-13-(2-methylbutanoyl)-6,7-epoxy-4,5,9,12,13,20-hexahydroxy-1-tigliaen-3-one (compound 1) in high yield and/or substantially pure form.

In one aspect of the present invention there is provided 12,13-dihexanoyl-6,7-epoxy-4,5,9,12,13,20-hexahydroxy-1-tigliaen-3-one (compound 2) or 12-tigloyl-13-(2-methylbutanoyl)-6,7-epoxy-4,5,9,12,13,20-hexahydroxy-1-tigliaen-3-one (compound 1) produced by the method as set out above.

In yet a further aspect of the present invention, there is provided a process for making a crystalline form of a compound of formula (I):

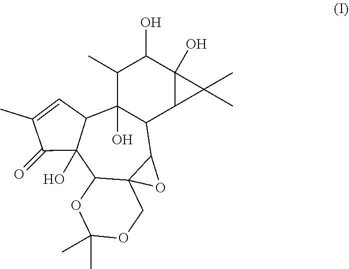

comprising crystallizing the compound of formula (I) from acetonitrile or methanol solvent.

### EXAMPLES

### Example 1—Extraction of Biomass

9.49 kg of ground seeds of Fontainea picrosperma (Analysis: Total tiglianone compounds: 6.7%, 635.8 g) were suspended in methanol (24 L) and the obtained suspension was maintained under stirring at room temperature for 4 hours. The suspension was allowed to stand for 2.5 hours and then the methanolic phase was removed and collected. The residual biomass was re-extracted twice more following the same procedure then washed once with 10 L of methanol with a settling time of 10 minutes. The combined methanolic extracts were concentrated under vacuum at a temperature of less than 40° C. to a final volume 20 L and the crude methanolic solution was analyzed by High Performance Liquid Chromatography (HPLC) and Karl Fischer (KF) titration. HPLC was performed using a reverse phase amide column (Halo P/N 92814-707) and guard column (Halo P/N 92814-107) with Solvent A: Water/0.1% formic acid and Solvent B: acetonitrile/0.1% formic acid with a linear gradient:

| Time (min) | Solvent A (%) | Solvent B (%) |
|---|---|---|
| 0.0 | 90 | 10 |
| 20.0 | 5 | 95 |
| 24.0 | 5 | 95 |
| 24.1 | 90 | 10 |
| 28.0 | 90 | 10 |

The flow rate was 1. mL/min, injection volume 5.0 μL, column temperature 40° C. and detection at 249 nm.

Analysis is shown in Table 1:

TABLE 1

| | | Total epoxytigliane | Epoxytigliane |
|---|---|---|---|
| | Water Content | compounds by | compound recovery |
| Composition | (KF) | HPLC | % |
| 20 L concentrated | 12.1% | 704.2 g | 110.8% |
| MeOH solution | | | |

### Example 2—Crude Extract Purification

The crude concentrated methanolic solution from Example 1 was extracted twice with n-hexane (30 L for the 1^{st} extraction, 15 L for the 2^{nd} extraction) at room temperature. Each extraction was mixed for about 10 minutes and then the phases allowed to separate for 10 minutes before the hexane phase was collected. The collected hexanic phases were combined and re-extracted twice with a methanol/water (90/10) mixture and then discharged. The combined hydro-methanolic phase was pooled with the previous methanol extractions and was diluted with 10% NaCl (aq, 5 L). The aqueous methanolic solution was extracted three times with dichloromethane (10 L for the 1^{st} extraction, 5 for the 2^{nd} and 3^{rd} extractions). The combined organic phases were dried over Na₂SO₄ and then concentrated under vacuum at a temperature of less than 40° C. until an oil having less than 0.1% water content as measured by KF was obtained.

A further batch of purified crude extract was prepared using the above-described process except that the combined hydromethanolic phase with pooled with previous methanol extractions and diluted with water rather than 10% NaCl, in order to reduce chloroadduct formation.

### Example 3—Preparation of Epoxytigliane-5,20 Acetonide Esters

The oil from Example 2 was dissolved in acetone (4 L); then, 2,2-dimethoxypropane (1.125 L) and pyridinium p-toluenesulfonate (0.338 Kg) were added. The mixture was heated under stirring at 40° C. for 25 hours. A sample was then checked by HPLC analysis as described in Example 1 to confirm the completion of the reaction. Ethyl acetate was added to the reaction mixture and the reaction mixture was then washed twice with water. The collected organic phases were dried under vacuum to yield the 5,20-acetonide esters as an oil.

### Example 4—Preparation of De-Esterified Epoxytigliane-5,20-Acetonide: Compound of Formula (Ia)

The oil from Example 3 was dissolved in methanol (10 L) and 1 kg of Cs₂CO₃ was added. The mixture was maintained under stirring at 25° C. for 22 hours, after which water (2 L) was added. The resulting mixture was washed three times with n-hexane (3×3 L). Ethyl acetate (10 L), water (8 L) and H₂SO₄ 2 N solution (5 L) were added to the residual hydro-methanolic phases. The organic phase was collected and the hydro-methanolic solution was re-extracted with ethyl acetate (10 L×4 times), pooling together all the ethyl acetate organic solutions. The ethyl acetate solution was then washed twice with 10% Na₂SO₄ aq. solution (2 L×2), discharging the water phase every time, and then was concentrated under vacuum until biphasic mixture was obtained. After phase separation, the water phase was discharged and the organic phase was concentrated again to give the to give the 12,13-dihydroxy acetonide compound of formula (II) as an oil. The oil was purified by column chromatography on silica gel (n-hexane/ethyl acetate 20:80 as eluent) to afford 163 g of compound of 81.3% purity as measured by HPLC.

### Example 5—Preparation of 12,13-dihexanoyl-6,7-epoxy-4,5,9,12,13,20-hexahydroxy-1-tigliaen-3-one-5,20-acetonide

To a solution of the product of Example 4 in dichloromethane (5 L), 27.8 g of 4-DMAP and 132.0 g of hexanoic acid were added and the mixture obtained was stirred until dissolution. 216.8 g of ethylcarbodiimide hydrochloride (EDC HCl) was added and the mixture was maintained stirring at 25° C. for 24 h. The reaction mixture was worked-up by adding dropwise 3.3 L of H₂SO₄ 2N and, after phase separation, the water phase was eliminated. The organic residual solution was washed with water (3.3 L×3) and then concentrated to dryness to give the dihexanoyl product.

A further batch of 12,13-dihexanoyl-6,7-epoxy-4,5,9,12,13,20-hexahydroxy-1-tigliaen-3-one-5,20-acetonide was prepared using the above-described process except that, prior to addition of H₂SO₄, a preliminary water washing step was carried out, to reduce chloroadduct formation.

### Example 6—Preparation of 12,13-dihexanoyl-6,7-epoxy-4,5,9,12,13,20-hexahydroxy-1-tigliaen-3-one (compound 2)

To a solution of the product of Example 5 in acetonitrile (3.1 L), an acidic aqueous solution containing HClO₄ 70% (300 mL of water+12.4 g of HClO₄ 70%) was added and the obtained mixture was stirred at 25° C. for 47 hours, monitoring reaction completion by thin layer chromatography (eluent MeCN/H₂O 85:15 v/v, silica gel, 60 RP18F 254S). The reaction was worked-up by adding 5 L of dichloromethane and 900 mL of a sodium acetate 10% aq. solution. The water phase was discharged and the organic solution was washed twice with water (2 L×2) then concentrated to dryness, yielding crude 12,13-dihexanoyl-6,7-epoxy-4,5,9,12,13,20-hexahydroxy-1-tigliaen-3-one.

The crude product was purified by column chromatography on reverse phase RP18 (acetonitrile/water 50:50+20 ppm H₃PO₄ as eluent). Only the central fractions containing high amounts of desired compound were pooled to reduce the presence of a hydrochloride adduct impurity believed to be:

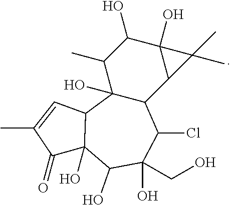

The impurity compound was analyzed by LC-HRMS (sodium salt) C₃₂H₄₉O₁₀Cl Na=651.2906.

The pooled fractions were diluted with water and extracted three times with dichloromethane. The combined organic phases were concentrated to dryness to give 93.7 g (57% from 10 Kg of undried seeds) of pure 12,13-dihexanoyl-6,7-epoxy-4,5,9,12,13,20-hexahydroxy-1-tigliaen-3-one.

A further batch of 12,13-dihexanoyl-6,7-epoxy-4,5,9,12,13,20-hexahydroxy-1-tigliaen-3-one (compound 2) was prepared using the above-described process except that, prior to final concentration to dryness of 12,13-dihexanoyl-6,7-epoxy-4,5,9,12,13,20-hexahydroxy-1-tigliaen-3-one, a water-washing step is carried out on concentrated 12,13-dihexanoyl-6,7-epoxy-4,5,9,12,13,20-hexahydroxy-1-tigliaen-3-one to prevent formation of an impurity.

### Example 7—Crystallization Study

Early intermediates in the synthetic pathway for producing compound 2, such as the compound of a formula (I), were investigated for crystallization potential.

An aliquot of purified compound of formula (Ia) obtained from Example 4 (0.83 L containing about 10 g of compound) was used in the crystallization study. The result of preliminary screening is shown in Table 2:

TABLE 2

| | Solvent | | | |
|---|---|---|---|---|
| | Volume vs | | | Crystallization |
| Solvent | Formula (Ia) | T ° C. | Comment | Result |
| Acetone | 5 V → 10 V | 20-25° C. → | Soluble only | No |
| | | reflux | with 10 V of | crystallization |
| | | | acetone at | observed even |
| | | | reflux | upon cooling to |
| | | | | 0° C. |
| Dimethylformamide | 5 V → 10 V | 20-25° C. | Soluble only | No |
| | | | with 10 V of | crystallization |
| | | | DMF | observed |
| Acetone/ | 10 V | reflux | Jelly precipitate | No |
| n-hexane | 10 V → 20 V | | observed after | crystallization |
| | | | addition of n- | observed |
| | | | hexane | |
| Methyl tert-butyl | 5 V → 20 V | 20-25° C. → | Not soluble | No |
| ether | | reflux | | crystallization |
| | | | | observed |
| Ethyl acetate | 5 V → 10 V | 20-25° C. | Remained | No |
| | | | dissolved | crystallization |
| | | | | observed even |
| | | | | upon cooling to |
| | | | | 0° C. |
| Ethyl acetate/ | 10 V | 20-25° C. | Jelly precipitate | No |
| n-hexane | 10 V | | observed after | crystallization |
| | | | addition of n- | observed |
| | | | hexane | |
| 2-Propanol | 5 V → 10 V | 20-25° C. → | Remained | No |
| | | reflux | dissolved | crystallization |
| | | | | observed even |
| | | | | upon cooling to |
| | | | | 0° C. |
| Methanol | 6 V | 20-25° C. | | Crystallization |
| | | | | observed after |
| | | | | dissolution |
| Acetonitrile | 5 V → 10 V | 20-25° C. → | Dissolution | Crystallization |
| | | reflux | observed at | observed at T |
| | | | reflux | about 70° C. |

Crystallization in methanol and acetonitrile were investigated further.

### Example 8—Crystalline Compound (Ta) as MeOH Solvate

A sample of the product of Example 4 (amorphous solid, with HPLC purity of about 80-85% was dissolved in methanol at room temperature; complete dissolution occurred in a few seconds. Crystallization was triggered without control immediately after dissolution. The product obtained was a solvate crystalline form with a MeOH content around 5-6%. In the following Table 3 the crystallization trials carried out are summarized:

TABLE 3

| Volume of | | | | |
|---|---|---|---|---|
| MeOH | | | | |
| (vs weight in g of | | Compound of formula | | |
| Compound of | T | (Ia) A % | | |
| formula Ia) | (° C.) | crude | crystallized | Yield |
| 3 V | 20-25° C. | 83.8% | 99.6% | 81% |
| 3.5 V | 20-25° C. | 79.4% | 98.8% | 81% |
| 3 V | 20-25° C. | 87.6% | 99.1% | 70% |

Crystalline methanol solvate of Compound of formula (Ia) was characterized by an X-ray powder diffraction (X-RPD) pattern obtained using the copper wavelengths λ₁ and λ₂ of 1.54056 Å and 1.54439 Å showing a crystalline structure and comprising distinctive reflections, expressed as 20 degrees values, at 7.2, 9.5, 11.5, 12.6, 13.1, 14.0, 14.5, 17.0, 17.7, 17.9, 19.2, 20.3, 21.2, 22.8, 23.7, 24.7, 27.1, 28.3, 29.7, 32.6 2θ.

The Fourier-Transform InfraRed Spectroscopy (FTIR) spectrum for the crystalline methanol solvate in the 4000-500 cm⁻¹ spectral range in ATR mode comprised characteristic absorption frequencies at approximately 3443.1, 3374.1, 3189.1, 3002.6, 2975.7, 2944.4, 2863.6, 1703.3, 1635.7, 1461.9, 1372.9, 1290.7, 1221.1, 1077.1, 1025.1, 992.0, 973.9, 929.7, 905.8, 876.7, 829.0, 769.1 cm⁻¹.

A Differential Scanning Calorimetry (DSC) profile of the crystalline methanol solvate is characterized by an endothermic peak with onset at about 145.33° C. and two other less intense endothermic peaks with onset at about 221.12° C. and 234.22° C., respectively.

The crystalline methanol solvate is further characterised by a Thermogravimetric profile (TG) showing a weight loss of 5.5%, which is consistent with the presence of 5-6% of residual MeOH.

### Example 9—Anhydrous Crystalline Compound of Formula (Ia)

A mixture of the product of Example 4 and acetonitrile was stirred under reflux until a clear solution was obtained; then the solution was allowed to cool where crystallization spontaneously occurred upon cooling at about 68 to 70° C. In the following Table the crystallization trials carried out are summarized:

| Volume of | | | | |
|---|---|---|---|---|
| Acetonitrile | | | | |
| (vs weight in g of | | Compound of formula | | |
| Compound of | T | (Ia) A % | | |
| formula Ia) | (° C.) | crude | crystallized | Yield |
| 10 V | reflux till | 87.1% | 99.9% | 75% |
| | dissolution → | | | |
| | 20-25° C. for the | | | |
| | filtration | | | |
| 7 V | reflux till | 87.1% | 98.7% | 84% |
| | dissolution → | | | |
| | 20-25° C. for the | | | |
| | filtration | | | |
| 6 V | reflux till | 87.1% | 99.9% | 87% |
| | dissolution → | | | |
| | 0-5° C. for the | | | |
| | filtration | | | |

Upon crystallization from acetonitrile, a solid was formed that was a 1:1 MeCN solvate. The acetonitrile solvate was collected and dried to provide Form A of an anhydrous crystalline compound of formula (I). The MeCN solvate had a Loss on Drying (LoD) of 7.8% wt. The MeCN solvate was characterized by an X-ray powder diffraction (X-RPD) pattern obtained using the copper wavelengths λᵢ and λ₂ of 1.54056 Å and 1.54439 Å showing a crystalline structure and comprising distinctive reflections, expressed as 20 degrees values, at 6.2, 7.5, 10.4, 12.2, 14.9, 16.0, 17.7, 18.5, 19.5, 20.9, 22.4, 24.7, 26.8 and 33.4 degrees 2θ±0.2 2θ.

Form A of a crystalline anhydrous Compound of formula (Ia) was characterized by an X-ray powder diffraction (X-RPD) pattern obtained using the copper wavelengths λᵢ and λ₂ of 1.54056 Å and 1.54439 Å showing a crystalline structure and comprising distinctive reflections, expressed as 20 degrees values, at 6.2, 7.6, 10.5, 12.5, 15.2, 16.2, 16.7, 18.3, 19.9, 21.5, 23.3, 25.2 and 27.8 degrees 2θ±0.2 2θ.

Form A of the anhydrous crystalline Compound of formula (Ia) was further characterized by a water content of about 0.28% by Coulometric Titration and residual organic solvent determination of 34 ppm by Head Space Gas Chromatography with flame ionization detection (HS GC FID).

Form A of the crystalline anhydrous Compound of formula (Ia) was stable for at least 9 months in industrial packaging (Type III amber glass vial (10 mL) closed by a black plastic screw cap with polyethylene stoppers) at 25° C. and 60% relative humidity and as a free crystalline powder at 80° C. and 60% humidity for 24 hours.

Upon storage at 5° C. for 5 days Form A of the anhydrous crystalline compound was transformed into a dihydrate form of the compound having a Loss on Drying (LoD) of 9.3% wt. The dihydrate form could be transformed back to Form A of the anhydrous crystalline form upon storage at 25° C. and 80% relative humidity for 5 days.

The dihydrate crystalline form was characterized by an X-ray powder diffraction (X-RPD) pattern obtained using the copper wavelengths λ₁ and λ₂ of 1.54056 Å and 1.54439 Å showing a crystalline structure and comprising distinctive reflections, expressed as 20 degrees values, at 6.2, 7.4, 8.9, 9.9, 10.5, 12.4, 15.3, 18.6, 20.2, 21.1, 22.4, 22.9, 26.0, 27.0, 27.3 and 33.7±0.2 degrees 2θ.

Upon storage of the Form A anhydrous crystalline form at 25° C. at 80% relative humidity for 68 hours, or 40° C. at 80% relative humidity for 7 days, a new stable anhydrous crystalline form was formed, Form B. Form B had a LoD of 0.3% wt and was stable at 25° C. at 80% relative humidity for at least a further 7 days unchanged. The anhydrous crystalline Form B was characterized by an X-ray powder diffraction (X-RPD) pattern obtained using the copper wavelengths λ₁ and λ₂ of 1.54056 Å and 1.54439 Å showing a crystalline structure and comprising distinctive reflections, expressed as 20 degrees values, at 4.9, 8.5, 9.8, 11.4, 14.6, 16.5, 17.5, 19.5, 21.4, 27.7, 28.7±0.2 degrees 2θ.

### Example 10: Comparison with and without Purification by Crystallization

With the process described above for the preparation of Compound 2, analysis was performed to compare the preparation of Compound 2 via the compound of formula (Ia) with and without crystallization.

From batches of 9.5 kg of undried seeds, following the processes described above and after chromatography, about 165-176 g of Compound of formula Ia was obtained.

Without the crystallization step, and following removal of about 10 g of the Compound of formula Ia for other studies, processing of the Compound of formula Ia provided Compound 2 in an amount of 92 g and analysis by HPLC showed the product had a 97.5% purity.

With crystallization of the compound of formula (Ia), and following removal of about 4.7 g of the Compound of formula Ia for other studies, processing of the Compound of formula Ia provided Compound 2 in an amount of 152 g and analysis by HPLC showed 98.7% purity.

### Example 11: Preparation of Anhydrous Crystalline Compound of Formula (Ia)

Selected fractions from silica gel purification of the compound of formula (Ia) (160-190 g compound of formula Ia), had solvent exchanged for acetonitrile (12 volumes used) with bath temperature set at 40-45° C. and approximately 100 mbar vacuum. A mixture of compound of formula Ia was then heated to reflux (82° C.) in acetonitrile (10 volumes) until dissolved, and then cooled with stirring to 20±5° C. The resulting slurry was stored at 20±5° C. overnight, and was then cooled to 0±5° C. for at least one hour. The mixture was then filtered under vacuum through a sintered glass filter. The filter cake was washed with acetonitrile (1 volume). The solid was kept under vacuum whilst on the filter, then charged into a steel tray, and dried in a dryer at approximately 50° C. under vacuum. The yield of compound of formula Ia was in the range 138-167 g.

1-33. (canceled)

34. A crystalline form of a compound of formula (I): 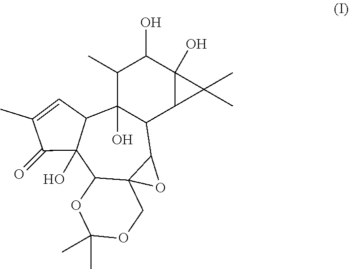
or a stereoisomer, pharmaceutically acceptable salt or solvate thereof.

35. The crystalline form of a compound of formula (I) according to claim 34, which is a compound of formula (Ia): 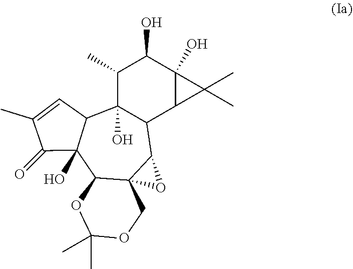
or a pharmaceutically acceptable salt or solvate thereof.

36. The crystalline form according to claim 35 in anhydrous form.

37. The crystalline form according to claim 36 that exhibits an X-ray powder diffraction pattern comprising a peak at about 10.5 degrees 2θ.

38. The crystalline form according to claim 36 that exhibits an X-ray powder diffraction pattern comprising peaks at about 6.2, 7.6 and 10.5 degrees 2θ.

39. The crystalline form according to claim 36 that exhibits an X-ray powder diffraction pattern comprising peaks at about 6.2, 7.6, 10.5, 12.5 and 15.2 degrees 2θ.

40. The crystalline form according to claim 36 that exhibits an X-ray powder diffraction pattern comprising peaks at about 6.2, 7.6, 10.5, 12.5, 15.2, 16.2, 18.3, 19.9, 23.2 and 27.8 degrees 2θ.

41. The crystalline form according to claim 36 that exhibits an X-ray powder diffraction pattern comprising a peak at about 11.4 degrees 2θ.

42. The crystalline form according to claim 41 that exhibits an X-ray powder diffraction pattern comprising peaks at about 8.5, 9.8 and 11.4 degrees 2θ.

43. The crystalline form according to claim 41 that exhibits an X-ray powder diffraction pattern comprising peaks at about 4.9, 8.5, 9.8, 11.4 and 14.6 degrees 2θ.

44. The crystalline form according to claim 36 that exhibits an X-ray powder diffraction pattern comprising peaks at about 4.9, 8.5, 9.8, 11.4, 14.6, 16.5, 17.5, 19.5, 21.4, 27.7 and 28.7 degrees 2θ.

45. The crystalline form according to claim 35 in the form of a methanol solvate.

46. The crystalline form according to claim 45 that exhibits an X-ray powder diffraction pattern comprising a peak at about 9.5 degrees 2θ.

47. The crystalline form according to claim 45 that exhibits an X-ray powder diffraction pattern comprising peaks at about 7.2, 9.5 and 13.1 degrees 2θ.

48. The crystalline form according to claim 45 that exhibits an X-ray powder diffraction pattern comprising peaks at about 7.2, 9.5, 11.5, 13.1 and 17.9 degrees 2θ.

49. The crystalline form according to claim 45 that exhibits an X-ray powder diffraction pattern comprising peaks at about 7.2, 9.5, 11.5, 12.6, 13.1, 14.0, 17.9, 20.3, 21.2, 22.8, and 32.6 degrees 2θ.

50. The crystalline form according to claim 35 which is a dihydrate crystalline form.

51. The crystalline form according to claim 50 that exhibits an X-ray powder diffraction (XRPD) pattern comprising at least one peak at about 10.5 degrees 2θ.

52. The crystalline form according to claim 50 that exhibits an XRPD pattern comprising peaks at about 10.5, 7.4 and 12.4 degrees 2θ.

53. The crystalline form according claim 50 that exhibits an XRPD pattern comprising peaks at about 10.5, 7.4, 12.4, 6.2, 9.9 and 15.3 degrees 2θ.

54. The crystalline form according to claim 50 that exhibits an XRPD pattern with peaks at about 6.2, 7.4, 8.9, 9.9, 10.5, 12.4, 15.3, 18.6, 20.2, 21.1, 22.4, 22.9, 26.0, 27.0, 27.3 and 33.7 degrees 2θ.

55. The crystalline form according to claim 35, wherein the compound of formula (Ia) is in substantially pure form.

56. A method of making a crystalline form of a compound of formula (I) comprising the steps of:
i) providing a mixture comprising one or more compounds of formula (II): 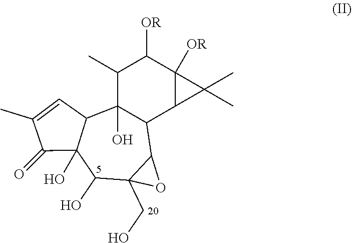
wherein each R is independently selected from H and —C(O)R₁, wherein when only one compound or formula (II) is present in the composition, at least one R group is not hydrogen; and
R₁ is selected from C₁-C₂₀alkyl, C₂-C₂₀alkenyl, C₂-C₂₀alkynyl, cycloalkyl, aryl, C₁₋₁₀alkylcycloalkyl; C₂₋₁₀alkenylcycloalkyl, C₂₋₁₀alkynylcycloalkyl, C₁₋₁₀alkylaryl, C₂₋₁₀alkenylaryl, C₂₋₁₀alkynylaryl, C₁₋₁₀alkylC(O)R₂, C₂₋₁₀alkenylC(O)R₂, C₂₋₁₀alkynylC(O)R₂, C₁₋₁₀alkylCH(OR₂)(OR₂), C(O)C₂₋₁₀alkenylCH(OR₂)(OR₂), C₂₋₁₀alkynylCH(OR₂)(OR₂), C₁₋₁₀alkylSR₂, C₂₋₁₀alkenylSR₂, C₂₋₁₀alkynylSR₂, C₁₋₁₀alkylC(O)OR₂, C₂₋₁₀alkenylC(O)OR₂, C₂₋₁₀alkynylC(O)OR₂, C₁₋₁₀alkylC(O)SR₂, C₂₋₁₀alkenylC(O)SR₂, C₂₋₁₀alkynylC(O)SR₂, 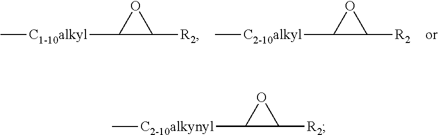
R₂ is hydrogen, —C₁₋₁₀alkyl, —C₂₋₁₀alkenyl, —C₂₋₁₀alkynyl, cycloalkyl or aryl;
wherein each alkyl, alkenyl, alkynyl, cycloalkyl or aryl group is optionally substituted;
ii) forming a 5,20-acetonide of formula (III): 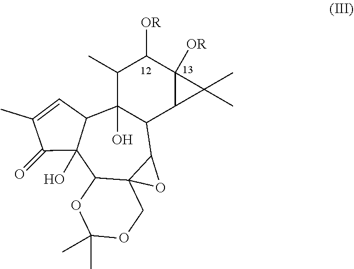
by treating the compound of formula (II) with 2,2-dimethoxypropane and a weakly acidic catalyst;
iii) de-esterifying the esters at C12 and C13 of formula (III) to provide a compound of formula (I): 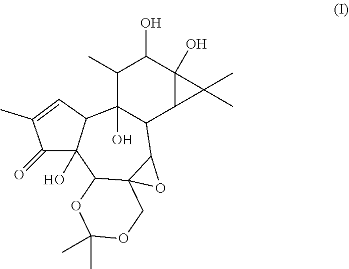
by treating the compound of formula (III) with a base; and
iv) crystallizing the compound of formula (I).

57. 12-tigloyl-13-(2-methylbutanoyl)-6,7-epoxy-4,5,9,12,13,20-hexahydroxy-1-tigliaen-3-one or 12,13-dihexanoyl-6,7-epoxy-4,5,9,12,13,20-hexahydroxy-1-tigliaen-3-one in substantially pure form.
